(19) United States
(12) Patent Application Publication
(10) Pub. No.: US 2023/0073288 A1
(43) Pub. Date: Mar. 9, 2023

(54) METHODS FOR MONITORING THE PROGRESS OF POLYSACCHARIDE SIZE-REDUCTION

(71) Applicants: Jennifer C. P. WANG, West Point, PA (US); Kelsey A. RICHARDS, Philadelphia, PA (US); Jayesh DESAI, Kenilworth, NJ (US); Bhumit A. PATEL, Kenilworth, NJ (US); Michael A. WINTERS, West Point, PA (US); Patrick M. MCHUGH, West Point, PA (US); Philip R. KUHL, West Point, PA (US); John P. HIGGINS, West Point, PA (US); Michael I. LARKIN, Santa Barbara, CA (US); Michelle H. CHEN, Goleta, CA (US); Merck Sharp & Dohme LLC, Rahway, NJ (US)

(72) Inventors: Jennifer C. P. Wang, Chesterbrook, PA (US); Kelsey A. Richards, Philadelphia, PA (US); Jayesh Desai, Paterson, NJ (US); Bhumit A. Patel, Edison, NJ (US); Michael A. Winters, Doylestown, PA (US); Patrick M. McHugh, Pipersville, PA (US); Philip R. Kuhl, Philadelphia, PA (US); John P. Higgins, Lansdale, PA (US); Michael I. Larkin, Santa Barbara, CA (US); Michelle H. Chen, Goleta, CA (US)

(73) Assignee: Merck Sharp & Dohme LLC, Rahway, NJ (US)

(21) Appl. No.: 17/800,271

(22) PCT Filed: Feb. 24, 2021
(86) PCT No.: PCT/US21/19364

§ 371 Date: Aug. 17, 2022

Related U.S. Application Data
(60) Provisional application No. 62/981,288, filed on Feb. 25, 2020.

(51) Int. Cl. A61K 39/09; C08B 37/00

(52) U.S. Cl. CPC A61K 39/092; C08B 37/00; A61K 2039/545

(57) ABSTRACT

The present invention relates to Polysaccharide Size-Reduction Monitoring Methods useful for the non-invasive, and accurate monitoring of the progress of a polysaccharide size-reduction process.

### FIELD OF THE INVENTION

The present invention relates to Polysaccharide Size-Reduction Monitoring Methods useful for the non-invasive, and accurate monitoring of the progress of a polysaccharide size-reduction process.

### BACKGROUND OF THE INVENTION

Polysaccharides are defined as polymers of monosaccharides linked by glycosidic bonds and exhibiting a degree of polymerization higher than 10. These macromolecules, reaching molecular weights of sometimes several million Daltons, present a large structural variability and have been described as composed of neutral (pentoses and hexoses) and/or anionic monosaccharides, sometimes substituted by non-sugar compounds. These structural features lead to specific behaviours and different architectures in solution, resulting in spirals, sheets, and also single, double and triple helices. The molar-mass distribution of polysaccharides is the primary parameter influencing their physicochemical properties in solution. For this reason, it is important to be able to achieve uniformity of the molar-mass distribution of polysaccharides, and to control their average molar mass and range of mass distribution. Methods for controlling the size of polysaccharides are primarily degradation-based, and include enzymatic, chemical and physical methods.

An important need for control over size distribution of polysaccharides is in the field of immunology, particularly in the use of polysaccharides in conjugate vaccines or immunogenic compositions. Carbohydrates in the form of capsular polysaccharides and/or lipopolysaccharides are the major components on the surface of bacteria. These molecules are important virulence factors in many bacteria isolated from infected persons.

Polysaccharide protein conjugate vaccines or immunogenic compositions (hereinafter referred to as “polysaccharide conjugate vaccines or immunogenic compositions”) consist of polysaccharides, generally from the surface coat of bacteria, linked to protein carriers. The combination of the polysaccharide and protein carrier induces an immune response against bacteria displaying the polysaccharide contained within the vaccine or immunogenic composition on their surface, thus preventing disease.

Determination of the molecular size distribution of vaccine products and immunogenic composition products is important during the vaccine and immunogenic composition manufacturing process. Molecular size distribution of conjugate vaccines and immunogenic compositions is a readily identifiable parameter that directly correlates with the immunogenicity. In particular, the efficacy of a polysaccharide conjugate vaccine or immunogenic composition is dependent on specific immunochemical and physical properties. The molecular size of the polysaccharide is one of the major parameters which can be directly correlated with vaccine or immunogenic composition immunogenicity and potency. This parameter is directly involved in inducing long-lasting immune memory. Determination of the molecular size distribution of vaccine products or immunogenic composition during manufacturing steps is crucial for routinely monitored batch-to-batch consistency.

Accordingly, there is a need for improved methods whereby one can accurately and continuously monitor the progress of polysaccharide size-reduction process. The present invention addresses that need.

### SUMMARY OF THE INVENTION

The present invention provides methods useful for the non-invasive, and accurate monitoring of the progress of a polysaccharide size-reduction process (the “Polysaccharide Size-Reduction Monitoring Methods”).

Accordingly, in one aspect, the present invention provides a first method for monitoring the size-reduction of polysaccharides, wherein said first method comprises the steps:
i) subjecting the polysaccharides to a size-reduction process, wherein the polysaccharides are in an aqueous solution during the size-reduction process;
ii) transferring a sample of the aqueous solution from step i to an instrument that can analyze the molecular weight of the polysaccharides in the aqueous solution;
iii) measuring the average molecular weight of the polysaccharides in the sample using the instrument; and
iv) optionally subjecting a subsequent sample of the aqueous solution from step i, to steps ii, iii, and iv.

The Polysaccharide Size-Reduction Monitoring Method can be useful, for example, for monitoring the progress of polysaccharide size-reduction in order to achieve a desired size distribution of polysaccharide molecules. Such control over size distribution can be useful in the field of immunology, where polysaccharide size control is critical during the manufacturing of polysaccharide conjugate vaccines and immunogenic products.

Accordingly, the present invention provides methods useful for the non-invasive and accurate monitoring of the progress of a polysaccharide size-reduction process.

The details of the invention are set forth in the accompanying detailed description.

Although any methods and materials similar to those described herein can be used in the practice or testing of the present invention, illustrative methods and materials are now described. Other embodiments, aspects and features of the present invention are either further described in or will be apparent from the ensuing description, examples and appended claims.

### BRIEF DESCRIPTION OF THE DRAWINGS

FIG. 1 shows a graphical description of the results of the procedure described in Example 1. The x-axis represents the timeline of the size-reduction process (in minutes), and the y-axis represents the average molecular weight (in kDa) of the polysaccharides that are being subjected to the size-reduction process.

FIG. 2 shows a graphical description of the results of the procedure described in Example 2. The x-axis represents the timeline of the size-reduction process (in minutes), and the y-axis represents the average molecular weight (in kDa) of the polysaccharides that are being subjected to the size-reduction process.

FIG. 3 shows a graphical description of the results of the procedure described in Example 3. The x-axis represents the timeline of the size-monitoring process (in minutes), and the y-axis represents the average molecular weight (in kDa) of the polysaccharides that are being subjected to the size-reduction process.

FIG. 4 shows an illustration of the at-line process described in Example 3. A solution of polysaccharide is size-reduced in vessel 1. A sample is manually taken from the active size-reduction process, and mixed with a buffer solution stored in vessel 3. The resulting buffered sample solution is then pumped through filter 4 via pump 2, and into the MALS instrument 5 for molecular weight calculation. The sample is then flushed from the MALS instrument into a waste container 6.

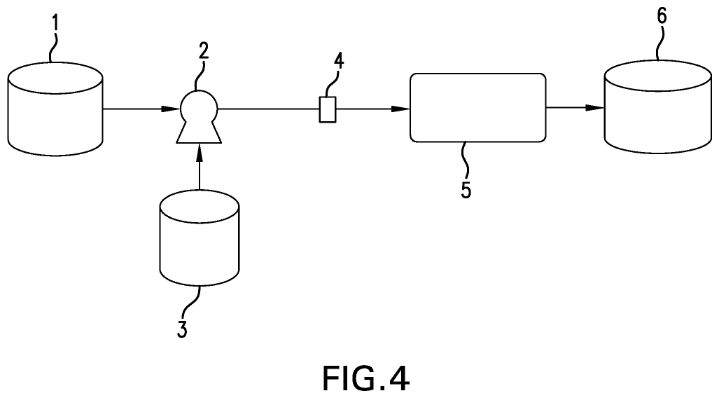

FIG. 5 shows an illustration of the on-line process described in Examples 1 and 2. A vessel 10, containing a solution of polysaccharide, is cycled through a homogenizer 9. During the homogenization process, batch samples are taken directly from the vessel using pump 2. The batch sample solution is then pumped through filter 7 and into the MALS instrument 8 for molecular weight calculation. The sample is then flushed into a waste container 6 using buffer solution stored in vessel 3.

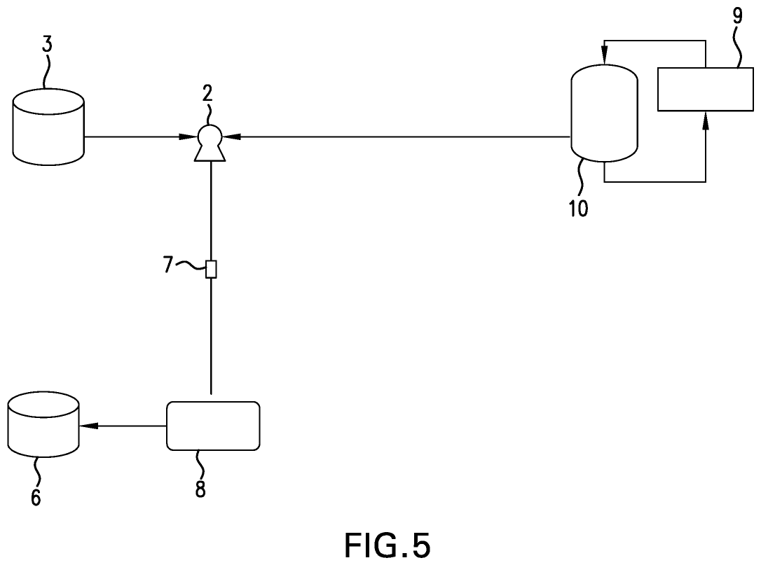

FIG. 6A shows a molar mass chromatogram of samples tested at various times during the size-reduction process described in Example 4, using the On-line LC-UV-MALS-RI system. The x-axis represents time in minutes, and the y-axis represents molar mass in g/mol. Line 11 represents the molar mass measurement taken at time T=0 of the size-reduction process; line 12 represents the molar mass measurement taken at time T=30 minutes of the size-reduction process; and line 13 represents the molar measurement taken at time T=210 minutes of the size-reduction process. FIG. 6B shows three overlapped refractive index chromatograms of samples tested at various times during the size-reduction process described in Example 4, using the On-line LC-UV-MALS-RI system. The x-axis represents time in minutes, and the y-axis represents the normalized relative scale of the overlapping lines to each other and has no units. Line 14 represents the liquid chromatogram taken at time T=0 of the size-reduction process; line 15 represents the liquid chromatogram taken at time T=30 minutes of the size-reduction process; and line 15 represents the liquid chromatogram taken at time T=210 minutes of the size-reduction process.

FIG. 7 shows a scatter plot of the size-reduction process described in Example 4, using the On-line LC-UV-MALS-RI system. This plot demonstrates the changes in the average molecular weight of the polysaccharide throughout the size-reduction process. The x-axis represents the timeline of the size-reduction process (in minutes), and the y-axis represents the average molecular weight (in kDa) of the polysaccharides in the solution that are being subjected to the size-reduction process. Data point 17 represents the molecular weight measurement taken at time T=0 of the size-reduction process; data point 18 represents the molecular weight measurement taken at time T=30 minutes of the size-reduction process; and data point 19 represents the molecular weight measurement taken at time T=210 minutes of the size-reduction process.

FIG. 8 shows a scatter plot of the size-reduction process described in Example 5, using the At-line LC-UV-MALS-RI system. This demonstrates the changes in molecular weight of the serotype throughout the size-reduction process. The x-axis represents the timeline of the size-reduction process (in minutes), and the y-axis represents the average molecular weight (in kDa) of the polysaccharides in the solution that are being subjected to the size-reduction process. Data point 17 represents the molecular weight measurement taken at time T=0 of the size-reduction process; data point 18 represents the molecular weight measurement taken at time T=30 minutes of the size-reduction process; and data point 19 represents the molecular weight measurement taken at time T=210 minutes of the size-reduction process.

FIG. 9 shows an illustration of the On-line LC-MALS-RI process described in Example 4. A solution of polysaccharide is size-reduced in vessel 23. During the size-reduction process, batch samples are taken directly 24 to the On-line LC-MALS-RI system, using a pump in the On-line LC-MALS-RI system. The sample is subjected to a chromatographic separation in the LC unit 25, then sequentially passed through the MALS instrument 26, a refractive index detector 27, and into a waste container 28. The information from the MALS instrument is fed (29) to a computer 31, and the information from the RI detector is also fed (30) to the computer 31, and the computer utilizes this information to determine the average molecular weight of the polysaccharides in the batch sample.

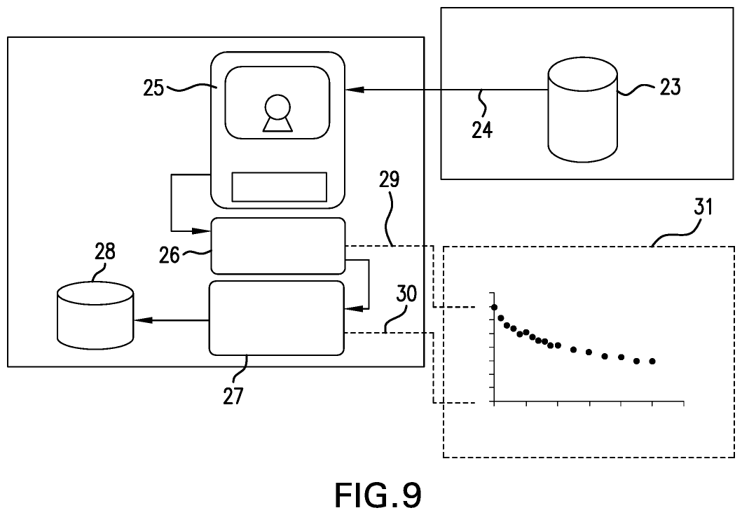

### DETAILED DESCRIPTION OF THE INVENTION

### Definitions and Abbreviations

The terms used herein have their ordinary meaning and the meaning of such terms is independent at each occurrence thereof.

The term “at-line” as used herein, refers to Polysaccharide Size-Reduction Monitoring Methods of the present invention, wherein the batch samples are manually taken and measured during the size-reduction process.

The term “At-Line LC-RI-MALS” as used herein, refers to a liquid chromatography (LC) system with a MALS and RI detector that is not physically connected to the vessel where the size reduction process is taking place, and wherein batch samples are manually drawn from the size-reduction process vessel, and delivered to the LC-RI-MALS system for analysis.

The term “BisTris,” as used here, refers to an organic: tertiary amine buffer with labile protons having a pKa of 6.46 at 25° C.

The term “homogenization” as used herein, means size-reduction.

The term “MALS” as used herein, means multi-angle light scattering.

The term “on-line” as used herein, refers to Polysaccharide Size-Reduction Monitoring Methods of the present invention, wherein the batch samples are taken as part of an inline process and measured during the size-reduction process.

The term “On-Line LC-RI-MALS” as used herein, refers to a liquid chromatography (LC) system with a MALS and RI detector, wherein said LC system is physically connected to the vessel where the size reduction process is taking place, and wherein batch samples can be automatically drawn from the size-reduction process vessel, and automatically delivered to the LC-RI-MALS system for analysis.

The term “present methods” as used herein, is understood to refer to all methods described herein for monitoring the size-reduction of polysaccharides.

The term “RI” as used herein, means refractive index.

The term “RT-MALS” as used herein, means real-time multi-angle light scattering.

### The Polysaccharide Size-Reduction Monitoring Methods

The present invention provides methods useful for the for the non-invasive, and accurate monitoring of the progress of polysaccharide size-reduction (The “Polysaccharide Size-Reduction Monitoring Methods” or the “present methods.”)

Accordingly, in one aspect, the present invention provides a first method (the “first method”) for monitoring the size-reduction of polysaccharides, wherein said method comprises the steps:
i) subjecting the polysaccharides to a size-reduction process, wherein the polysaccharides are in an aqueous solution during the size-reduction process;
ii) transferring a sample of the aqueous solution from step i to an instrument that can analyze the molecular weight of the polysaccharides in the aqueous solution;
iii) measuring the average molecular weight of the polysaccharides in the sample using the instrument; and
iv) optionally subjecting a subsequent sample of the aqueous solution from step i, to steps ii, iii, and iv.

In one embodiment, for the first method, the steps i to iii are carried out consecutively in a single, in-line, continuous process.

In another embodiment, for the first method, the sample from step i is manually transferred the instrument of step ii.

In one embodiment, for the first method, the aqueous solution of step i further comprises a salt.

In another embodiment, for the first method, a salt is added to the aqueous solution after step i is complete, but prior to commencing step ii.

Salts useful in the first method, include, but are not limited to sodium chloride, potassium chloride, calcium chloride, magnesium chloride, potassium acetate, sodium acetate, and combinations thereof. Non-ionic isotonic agents including but not limited to sucrose, trehalose, mannitol, sorbitol and glycerol may be used as a salt substitute in the present methods.

In one embodiment, the salt is sodium chloride.

A salt or salt substitute, when used, is present in an amount from about 25 mM to about 500 mM. In another method, a salt or salt substitute, when used, is present in an amount from about 20 mM to about 170 mM

In one embodiment, the salt is NaCl, optionally present at a concentration from 20 mM to 170 mM.

In addition to salts and salt substitutes, the aqueous solution of step i can also optionally comprise BisTris or sodium azide.

In one embodiment, for the first method, the molecular weight measuring in step iii is conducted using high-performance size exclusion chromatography, multi-angle light scattering (MALS), or refractive index measurements.

In one embodiment, for the first method, the sample of the aqueous solution from step i is filtered prior to commencing step ii.

In one embodiment, for the first method, the measuring in step iii is conducted using MALS.

In another embodiment, for the first method, the measuring in step iii is conducted using On-Line LC-RI-MALS.

In another embodiment, for the first method, the measuring in step iii is conducted using RT-MALS.

In still another embodiment, for the first method, the measuring in step iii is conducted using high-performance size exclusion chromatography.

In another embodiment, for the first method, the measuring in step iii is conducted using refractive index measurements.

In another aspect, the present invention provides a second method (the “second method”) for the online monitoring of a continuous size-reduction process of polysaccharides, wherein the polysaccharides are in an aqueous solution, and wherein the online monitoring method comprises the following sequential steps:
i) subjecting the polysaccharides to a size-reduction process, wherein the polysaccharides are in an aqueous solution during the size-reduction process;
ii) transferring a sample of the aqueous solution of step i to a liquid chromatography system that performs a chromatographic separation of the polysaccharides in the sample; then
iii) transferring the chromatography eluent of step ii to a first instrument that measures the light scattering of each of the polysaccharide components in the sample;
iv) transferring the sample solution exiting the first instrument to a second instrument, wherein the second instrument measures the concentration of each of the polysaccharide components using the refractive index of the polysaccharides;
v) calculating the average molecular weight of the polysaccharides in the aqueous solution, wherein the method of calculation utilizes: (a) the light scattering information obtained in step iii, and (b) the refractive index information obtained in step iv; and
vi) optionally subjecting a subsequent sample of the aqueous solution from step i to steps ii, iii, iv, v and vi.

In one embodiment, for the Polysaccharide Size-Reduction Monitoring Methods of the present invention, the polysaccharide size-reduction process is carried out using a physical method.

In one embodiment, the physical method of polysaccharide size-reduction comprises the use of a sampling valve. In another embodiment, the physical method of polysaccharide size-reduction comprises the use of a chopper blade. In another embodiment, the physical method of polysaccharide size-reduction comprises the use of dynamic high-pressure homogenization (HPH). In still another embodiment, the physical method of polysaccharide size-reduction comprises the use of ultrasonication. In another embodiment, the physical method of polysaccharide size-reduction comprises the use of thin-screw extrusion.

In one embodiment, for the Polysaccharide Size-Reduction Monitoring Methods of the present invention, the polysaccharide size-reduction process is carried out using a chemical method.

In one embodiment, the chemical method of polysaccharide size-reduction comprises the use of an acid-hydrolysis process. Acids useful in such an acid-hydrolysis process include, but are not limited to HC1, acetic acid, hydrochloric acid, phosphoric acid, and citric acid. In one embodiment, the acid is acetic acid.

In one embodiment, when an acid-hydrolysis process is used, the concentration of acid used in the process is between about 20 mM and about 300 mM. In another embodiment, the concentration of acid used in the process is between about 50 mM and about 200 mM. In another embodiment, the concentration of acid used in the process is between about 100 mM and about 200 mM. In a further embodiment, the concentration of acid used in the process is about 100 mM. In another embodiment, the concentration of acid used in the process is about 200 mM.

In another embodiment, the chemical method of polysaccharide size-reduction comprises the use of a base-hydrolysis process. Bases useful in a base-hydrolysis process include, but are not limited to NaOH, potassium phosphate, sodium bicarbonate, and potassium bicarbonate.

In another embodiment for the Polysaccharide Size-Reduction Monitoring Methods of the present invention, the polysaccharide size-reduction process is carried out using an enzymatic depolymerization process. In one embodiment, the enzymatic depolymerization process comprises the use of a lyase or hydrolase.

In still another embodiment, for the Polysaccharide Size-Reduction Monitoring Methods of the present invention, the polysaccharide size-reduction process is carried out using a thermal or thermochemical method. In one embodiment, the thermal or thermochemical method comprises the use of jet-cooking. In another embodiment, the thermal or thermochemical method comprises the use of microwave irradiation.

In one embodiment, for the present methods, the manual or automated sample extraction and subsequent measuring is carried out at regular intervals. In one embodiment, the sample extraction and subsequent measuring is carried out at regular intervals. In another embodiment, the sample extraction and subsequent measuring is carried out every 30 seconds, every 1 minute, every 2 minutes, every 3 minutes, every 4 minutes, every 5 minutes, every 6 minutes, every 7 minutes, every 8 minutes, every 9 minutes, every 10 minutes, every 20 minutes, every 25 minutes, every 30 minutes or every 1 hour. In another embodiment, the sample extraction and subsequent measuring is carried out every 1 minute. In another embodiment, the sample extraction and subsequent measuring is carried out every 5 minutes. In still another embodiment, the sample extraction and subsequent measuring is carried out every 10 minutes. In another embodiment, the sample extraction and subsequent measuring is carried out every 20 minutes. In another embodiment, the sample extraction and subsequent measuring is carried out every 25 minutes. In another embodiment, the sample extraction and subsequent measuring is carried out every 30 minutes.

In one embodiment, for the present methods, the sample extraction and subsequent measuring is performed from 1 to 40 times.

In another embodiment, for the present methods, the sample extraction and subsequent measuring is performed from 3 to 10 times.

In another embodiment, for the present methods, the sample extraction and subsequent measuring is performed from 5 to 7 times.

In still another embodiment, for the present methods, the sample extraction and subsequent measuring is performed from 10 to 20 times.

In another embodiment, for the present methods, the sample extraction and subsequent measuring is performed about 5 times.

In another embodiment, for the present methods, the sample extraction and subsequent measuring is performed about 10 times.

In a further embodiment, for the present methods, the sample extraction and subsequent measuring is performed about 15 times.

In another embodiment, for the present methods, the sample extraction and subsequent measuring is performed about 20 times.

In one embodiment, for the present methods, the resulting final average molecular weight of the bacterial surface polysaccharides is from 10-1000 kDa.

In another embodiment, for the present methods, the resulting final average molecular weight of the bacterial surface polysaccharides is from 100-800 kDa.

In another embodiment, for the present methods, the resulting final average molecular weight of the bacterial surface polysaccharides is from 200-650 kDa.

In another embodiment, for the present methods, the resulting final average molecular weight of the bacterial surface polysaccharides is from 90-500 kDa.

In another embodiment, for the present methods, the resulting final average molecular weight of the bacterial surface polysaccharides is from 50-500 kDa.

In one embodiment, the polysaccharides that are size-reduced using the present methods are bacterial surface polysaccharides. In another embodiment, the polysaccharides are bacterial surface polysaccharides derived from pneumococcal bacteria.

Specific polysaccharides that can be size-reduced using the present methods include, but not limited to, Meningococcal polysaccharides, Pneumococcal polysaccharides, Hemophilus influenzae type b polysaccharide, Vi polysaccharide of Salmonnella typhi, and group B Streptococcus polysaccharides.

Bacterial capsular polysaccharides, particularly those that have been used as antigens, are suitable for use in present methods of the invention, and can readily be identified by methods for identifying immunogenic and/or antigenic polysaccharides. These bacterial capsular polysaccharides may, for example, be from N. meningitidis, particularly serogroups A, C, W135 and Y; S. pneumoniae, particularly from serotypes: 1, 2, 3, 4, 5, 6A, 6B, 6C, 7C, 7F, 8, 9N, 9V, 10A, 11A, 12F, 14, 15A, 15B, 15C, 16F, 17F, 18C, 19A, 19F, 20, 22F, 23A, 23B, 23F, 24F, 33F, 35B, 35F, or 38; S. agalactiae, particularly serotypes Ia, Ib, and III; S. aureus, particularly from S. aureus type 5 and type 8; Haemophilus influenzae Type b; Salmonella enterica Typhi Vi; and Clostridium difficile.

In one embodiment, the polysaccharide is a Streptococcus pneumoniae capsular polysaccharide.

Non-capsular bacterial polysaccharides may also be suitable for use in the invention. An exemplary non-capsular bacterial polysaccharide is the S. pyogenes GAS carbohydrate (also known as the GAS cell wall polysaccharide, or GASP). present methods of the

Non-bacterial polysaccharides may also be suitable for use in the present methods of the invention. For example, the invention may use glucans, e.g. from fungal cell walls. Illustrative glucans include, but are not limited to, laminarin and curdlan.

### Vaccine and Immunogenic Composition Conjugates

In another aspect, the present invention provides conjugate vaccines and immunogenic compositions comprising: i) bacterial surface polysaccharides that have been size reduced using the Polysaccharide Size-Reduction Monitoring Methods, and ii) a protein carrier.

In one embodiment, a conjugate vaccine or immunogenic composition of the present invention comprises: i) bacterial surface polysaccharides obtained according to the method of claim 1 or 2, and ii) a protein carrier, wherein the final average bacterial surface polysaccharide distribution is from 70-1000 kDa.

Protein carriers useful in the conjugate vaccines or immunogenic compositions of the present invention include, but are not limited to: a genetically modified cross-reacting material (CRM) of diphtheria toxin, and H. influenzae protein D (HiD); additional inactivated bacterial toxins such as diphtheria toxin (DT), Diphtheria toxoid fragment B (DTFB), TT (tetanus toxid) or fragment C of TT, pertussis toxoid, cholera toxoid (e.g., as described in International Publication No. WO 2004/083251), E. coli LT (heat-labile enterotoxin), E. coli ST (heat-stable enterotoxin), and exotoxin A from Pseudomonas aeruginosa; bacterial outer membrane proteins such as outer membrane complex c (OMPC), porins, transferrin binding proteins, pneumococcal surface protein A (PspA; See International Publication No. WO 02/091998), pneumococcal adhesin protein (PsaA), C5a peptidase from Group A or Group B Streptococcus, or Haemophilus influenzae protein D, pneumococcal pneumolysin (Kuo et al., 1995, Infect Immun 63; 2706-13) including ply detoxified in some fashion for example dPLY-GMBS (See International Publication No. WO 04/081515) or dPLY-formol, PhtX, including PhtA, PhtB, PhtD, PhtE and fusions of Pht proteins for example PhtDE fusions, PhtBE fusions (See International Publication Nos. WO 01/98334 and WO 03/54007); other proteins, such as ovalbumin, keyhole limpet hemocyanin (KLH), bovine serum albumin (BSA) or purified protein derivative of tuberculin (PPD), PorB (from N. meningitidis), PD (Haemophilus influenzae protein D (see, e.g., European Patent No. EP 0 594 610 B), or immunologically functional equivalents thereof, synthetic peptides (See European Patent Nos. EP0378881 and EP0427347), heat shock proteins (See International Application Publication Nos. WO 93/17712 and WO 94/03208), pertussis proteins (See International Publication No. WO 98/58668 and European Patent No. EP0471177), cytokines, lymphokines, growth factors or hormones (See International Publication No. WO 91/01146), artificial proteins comprising multiple human CD4+ T cell epitopes from various pathogen derived antigens (See Falugi et al., 2001, Eur J Immunol 31:3816-3824) such as N19 protein (See Baraldoi et al., 2004, Infect Immun 72:4884-7), iron uptake proteins (See International Publication No. WO 01/72337), toxin A or B of C. difficile (See International Patent Publication No. WO 00/61761), and flagellin (See Ben-Yedidia et al., 1998, Immunol Lett 64:9).

Other diphtheria toxin mutants can also be used as the carrier protein, such as CRM176, CRM197, CRM228, CRM45 (Uchida et al., 1973, J Biol Chem 218:3838-3844); CRM9, CRM45, CRM102, CRM103 and CRM107 and other mutations described by Nicholls and Youle in Genetically Engineered Toxins, Ed: Frankel, Maecel Dekker Inc, 1992; deletion or mutation of Glu-148 to Asp, Gln or Ser and/or Ala 158 to Gly and other mutations disclosed in U.S. Pat. Nos. 4,709,017 or 4,950,740; mutation of at least one or more residues Lys 516, Lys 526, Phe 530 and/or Lys 534 and other mutations disclosed in U.S. Pat. Nos. 5,917,017 or 6,455,673; or fragment disclosed in U.S. Pat. No. 5,843,711.

In one embodiment, the protein carrier is a genetically modified cross-reacting material (CRM) of diphtheria toxin.

In another embodiment, the protein carrier is tetanus toxoid.

In another embodiment, the protein carrier is meningococcal outer membrane protein complex.

In still another embodiment, the protein carrier is diphtheria toxoid.

In another embodiment, the protein carrier is H. influenzae protein D.

In another embodiment, the protein carrier is CRM197.

In another embodiment, the conjugate vaccine or immunogenic composition of the present invention comprises the purified capsular polysaccharide of 15 serotypes of Streptococcus pneumoniae (PCV-15) wherein the serotypes are 4, 6B, 9V, 14, 18C, 19F, 23F, 1, 3, 5, 6A, 7F, 19A, 22F and 33F

In another embodiment, the conjugate vaccine or immunogenic composition of the present invention comprises the purified capsular polysaccharide of 21 serotypes Streptococcus pneumoniae (PCV-21) wherein the serotypes are 3, 6A, 7F, 19A, 22F, 33F, 8, 10A, 11A, 12F, 15B, 15A, 23B, 24F, 35B, 16F, 17F, 20, 23A, 31, and 9N, wherein the 15B is de-O-acetylated.

In another embodiment, the conjugate vaccine or immunogenic composition of the present invention comprises the purified capsular polysaccharide of 24 serotypes of Streptococcus pneumoniae (PCV-24) wherein the serotypes are 4, 6B, 9V, 14, 18C, 19F, 23F, 1, 3, 5, 6A, 7F, 19A, 22F, 33F, 8, 10A, 11A, 12F, 15B, 15A, 23B, 24F, and 35B wherein the 15B is de-O-acetylated.

In another embodiment, the conjugate vaccine or immunogenic composition of the present invention comprises the purified capsular polysaccharide of 20 serotypes of Streptococcus pneumoniae (PCV-20) wherein the serotypes are 4, 6B, 9V, 14, 18C, 19F, 23F, 1, 3, 5, 6A, 7F, 19A, 22F, 33F, 8, 10A, 11A, 12F, and 15B,

In another embodiment, the conjugate vaccine or immunogenic composition of the present invention is V114.

In another embodiment, the conjugate vaccine or immunogenic composition of the present invention is V116.

In another embodiment, the conjugate vaccine or immunogenic composition of the present invention is V117.

### Uses of the Conjugate Vaccines and Immunogenic Compositions

In one aspect, the present invention provides a method for treating or preventing a disease in a patient, wherein the method comprises administering an effective amount of a conjugate vaccine or immunogenic composition of the present invention.

Diseases treatable or preventable using the conjugate vaccines or immunogenic composition of the present invention include, but are not limited to, Haemophilus influenzae type b (Hib) disease, Hepatitis B, Human papillomavirus (HPV), whooping cough, pneumococcal infections, meningococcal infections, streptococcal infections, chicken pox, and shingles.

In one embodiment, the present invention provides a method for treating or preventing a disease in a patient, wherein the disease is caused by Streptococcus pneumoniae, wherein the method comprises administering an effective amount of a conjugate vaccine or immunogenic composition of the present invention.

In another embodiment, the present invention provides a method for treating or preventing a disease in a patient, wherein the disease is caused by Streptococcus pneumoniae, and wherein the method comprises administering an effective amount of PCV15, specifically V114, or PCV21, specifically V116, or PCV24, specifically V117, or PCV20.

In another embodiment, the present invention provides a method for treating or preventing a disease in a patient, wherein the disease is caused by Streptococcus pneumoniae, and wherein the method comprises administering an effective amount of PCV21, specifically V116.

The Polysaccharide Size-Reduction Monitoring Methods may be useful, for example, for the production of polysaccharides of various sizes and size distributions in the food and fine chemicals industries, and for the production of polysaccharide conjugate vaccines and immunogenic compositions in the pharmaceutical industry.

### EXAMPLES

### Example 1

### On-Line MALS Procedure (with Salt)

S. pneumoniae Serotype 7F Purified Polysaccharide Powder (2.5 g) was dissolved in sterile distilled water (620 mL) to provide a solution having a final concentration of approximately 4.0 g/L. The resulting solution was mixed with a stir bar until all polysaccharide powder was in solution, and the solution was filtered through a 0.45 μM filter. A sample of the filtrate was then assayed using High Performance Chromatography-Ultraviolet-Multi Angle Light Scattering-Refractive Index detection (HPSEC-UV-MALS-RI) for a concentration result of 4.0 g/L.

Another sample of the filtrate (372 mL) was then diluted with an aqueous NaCl solution (5M, 18 mL) and sterile distilled water (198 mL) to a final concentration of 2.5 g/L of polysaccharide, and 150 millimolar NaCl (the “diluted filtrate/salt solution”). A homogenization unit (GEA PandaPlus Benchtop Homogenizer) was then prepared by flowing approximately 2 L of water though the unit, followed by a 10-minute recirculation with aqueous NaOH solution (0.5N), then a final flush with water (2 L). A Wyatt Dawn Multi Angle Light Scattering detector (“MALS detector”) was then flushed with water using an Agilent Infinity II pump for at least 30 minutes. The diluted filtrate/salt solution was then place in an appropriate-sized beaker and mixed using a stir bar Tubing to the inlet and outlet of the homogenizer were placed in the solution and fixed to prevent movement.

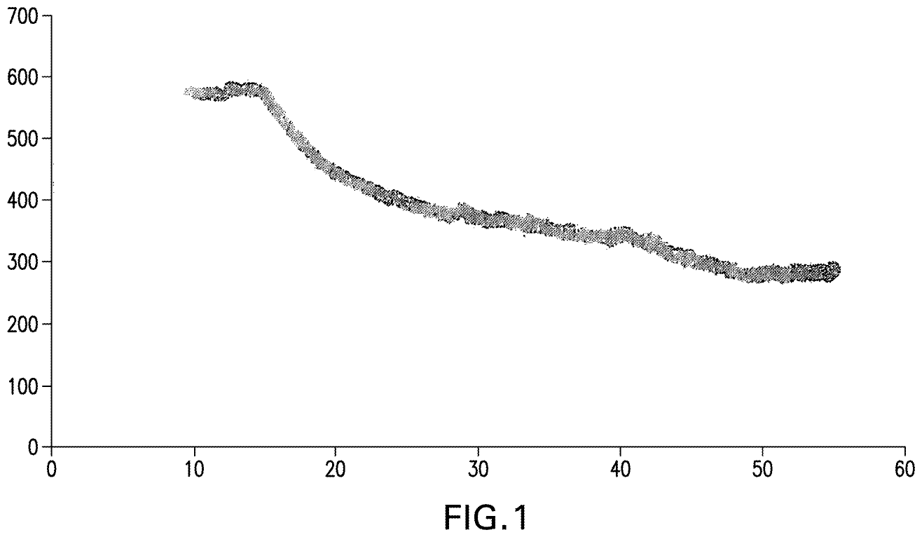

The pump was set to flow water through the MALS detector at 3 mL/minute. Using Wyatt Astra software, a baseline was collected for approximately 5 minutes. Detector collection and flow was paused while the tubing to the inlet of the Agilent pump was transferred from water to the diluted filtered dissolved polysaccharide solution. Flow was then restarted using the Agilent pump at 3 mL/minute, and data collection was resumed using the Wyatt Astra software. Once the MALS detector voltage was stabilized (indicated in the Astra software as an approximately flat line), the homogenizer is turned on and the batch is recirculated at 0 bar pressure though the homogenizer. A 5 mL Pass 0 sample is taken before pressurizing the homogenizer to 150 bar, with homogenization commencing when the target pressure is reached +/−10 bar. A single “pass” is measured as the amount of time it takes the entire batch volume to recirculate though the homogenizer (with batch volume measured in mL, and homogenizer flowrate measured in mL/minute). During each pass, a 5 mL sample is removed from the batch and the average molecular weight of the polysaccharide is measured. After 9 passes, the homogenizer was depressurized for approximately 2-5 minutes and then re-pressurized to 250 bar for further size-reduction. After 4 additional passes at 250 bar pressure, the homogenizer was depressurized to 0 bar and the batch was allowed to recirculate without size-reduction until the voltage signal on the MALS detector had stabilized. The homogenizer was then drained and water was used to flush residual sample from the system. Data collected using the Astra software was then be converted to molecular weight values using well-known methods. Results of this process are presented in graphical format in FIG. 1.

### Example 2

### On-Line MALS Procedure (without Salt)

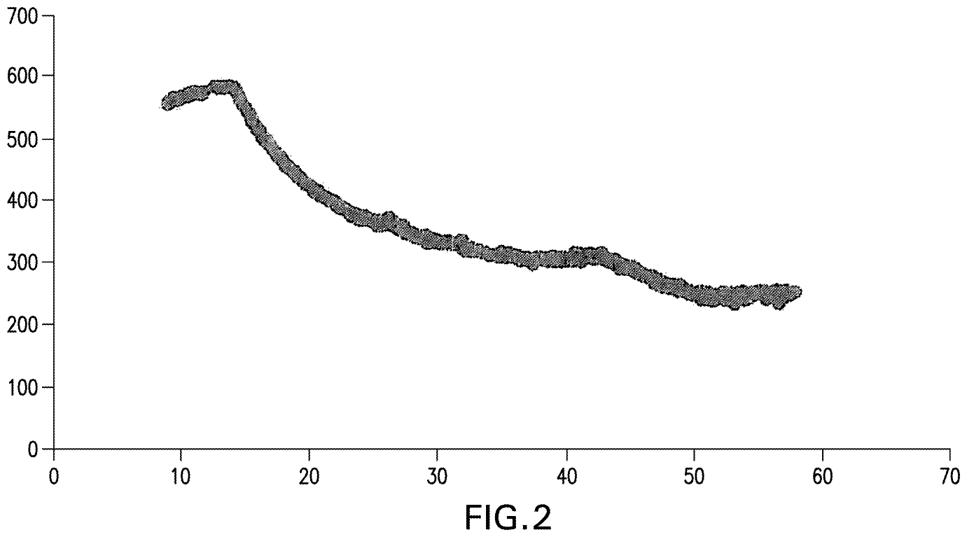

The procedure of Example 1 was followed exactly as described, except that sodium chloride was not added to the Filtered Dissolved Polysaccharide solution. Results of this process are presented in graphical format in FIG. 2.

### Example 3

### At-Line MALS Procedure

S. pneumoniae Serotype 6A Purified Polysaccharide Powder (15 g) was dissolved in sterile distilled water (3750 mL) to a final concentration of approximately 4.0 g/L. The resulting solution was mixed with a stir bar until all polysaccharide was fully dissolved, and the resulting solution was filtered through a 0.45 μM filter. A sample of the filtrate was assayed using High Performance Chromatography-Ultraviolet-Multi Angle Light Scattering-Refractive Index detection (HPSEC-UV-MALS-RI) for a concentration result of 4.0 g/L. Another sample of the filtrate (335 mL) was diluted from 4.0 g/L with 165 mL of sterile distilled water to a final concentration of 2.7 g/L of polysaccharide (the “diluted filtrate”). A homogenization unit (GEA Panda 2K Benchtop Homogenizer) was prepared by recirculating aqueous NaOH (0.5 N) for 30 minutes, then flushing with 2 L of water. The diluted filtrate sample was then put in an appropriately sized beaker and mixed using a stir bar. Tubing to the inlet and outlet of the homogenizer were placed in the solution and fixed to prevent movement.

A Wyatt Dawn HELEOS Multi Angle Light Scattering detector (“MALS detector”) was turned on, and flushed with water using an Agilent 1260 Quaternary pump for at least 30 minutes. The pump was set up with approximately 6-13 inches of tubing from sample container to Line B of the pump head. A Wyatt Aqueous Inline Filter Kit was installed between the pump and the MALS detector, using 0.22 μM filter paper. Both lines A and B of the pump were primed with “BisTris buffer” (aqueous solution of 10 mM BisTris and 150 mM NaCl buffer, pH 6.8). Using Wyatt Observer collection software, a baseline signal was collected by flowing BisTris buffer only through line A of the pump to the MALS detector at a flow rate of 2 mL/minute. After approximately 3 minutes, the software was paused, and flow was stopped though the pump.

The homogenization unit was then turned on and the batch was recirculated at 0 bar pressure though the homogenizer. A 5 mL Pass 0 sample is taken before pressurizing the homogenizer to 2000 bar, with homogenization commencing when the target pressure is reached +/−10 bar. A single “pass” is measured as the amount of time it takes the entire batch volume to recirculate though the homogenizer (with batch volume measured in mL, and homogenizer flowrate measured in mL/minute). At each pass, beginning with pass 2 (the second pass of the batch through the homogenizer), a 0.9 mL sample is removed from the batch. A 0.9 mL sample was also removed approximately halfway in-between passes 2 and 3, 3 and 4, 4 and 5, as well as passes 5 and 6. The batch sample is then homogenized for 7 passes, with molecular weight data being measured for each sample removed from the batch.

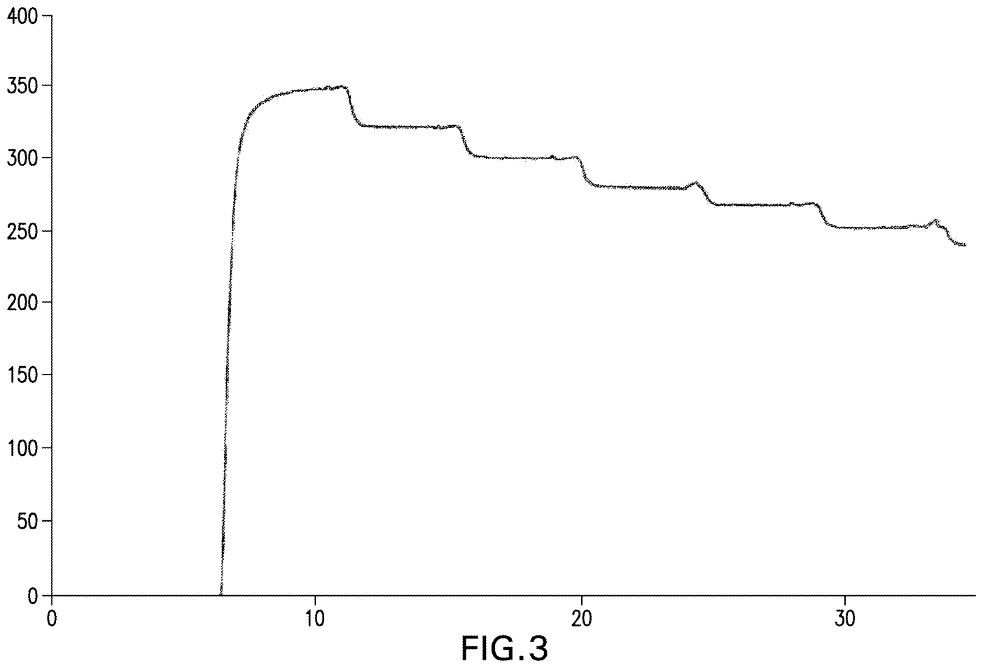

In parallel with the homogenization process, each of the collected 0.9 mL pass samples is mixed with 8.1 mL of BisTris buffer to target a 10% dilution of sample. The diluted sample is then vortexed for about 10 seconds before the Agilent line B is inserted into the tube. A 2 mL/minute flow is resumed using the HPLC Pump manager and the software is resumed. Once the signal in Observer has stabilized (as seen in the Astra software as an approximately flat line), the collection is paused until the next sample is ready. This sample dilution though collection process is repeated for each pass sample generated during homogenization. Data collected using the Astra software was then be converted to molecular weight values using well-known methods. Results of this process are presented in graphical format in FIG. 3.

The present invention is not to be limited by the specific embodiments disclosed in the examples that are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited herein, the entire disclosures of which are incorporated herein by reference.

### Example 4

### On-Line LC-MALS-RI Procedure

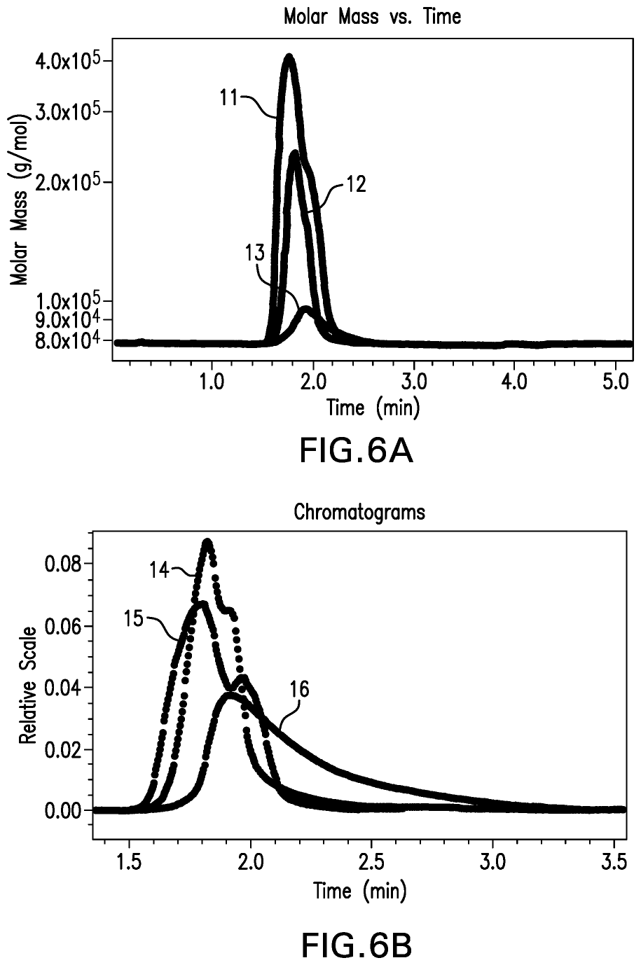

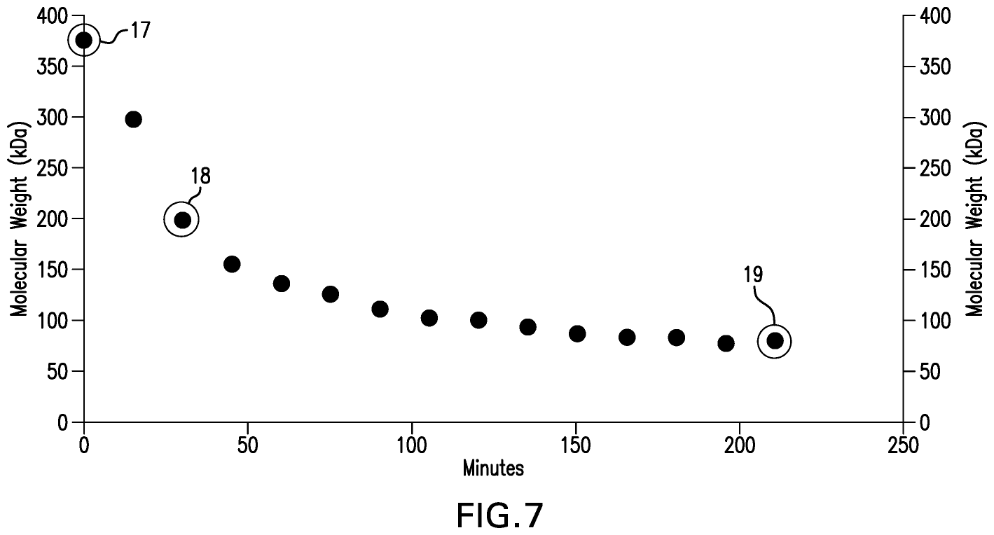

A solution of S. pneumoniae Serotype 6A in acetic acid (200 mM, solution concentration about 3.96 mg/mL) solution was introduced into a jacketed vessel having a jacket temperature of 80° C. and stirring was commenced to initiate an acid-hydrolysis based size reduction process. Prior to initiation of the polysaccharide solution, the vessel housing the size-reduction process was connected to an On-line LC-MALS-RI system using peek tubing. The On-line LC-MALS-RI system encompassed a Waters Patrol UPLC system (the “LC system”), a Wyatt uDawn MALS detector (the “MALS detector”), and a Wyatt Optilab UT-rEX RI detector(“RI detector”), which were connected in sequence. At initiation of the size-reduction process, the On-line LC-MALS-RI system performed an on-line injection (at time T=0 minutes) by drawing and delivering an aliquot of 1.5 mL from the size-reduction vessel in an automated fashion. The draw and delivery rate settings were at 1.0 mL/min. Subsequently, the on-line injection was chromatographically separated using size exclusion chromatography (SEC) using the LC system (flowrate for the SEC method was 0.5 mL/min, the temperature of the column was designated at 30° C., and the mobile phase was 10 mM BisTris and 150 mM NaCl buffer, at pH 6.8). Upon exiting the SEC column, the chromatography eluate was subsequently introduced into the MALS detector, where the multi-angle light scattering of the polysaccharides was measured. Upon exiting the MALS detector, the sample was introduced into the RI detector, where the refractive index of the polysaccharides was measured. Using Wyatt Astra software, the light scattering and refractive index signals were utilized to calculate the average molecular weight of the polysaccharides in the sample. Subsequent auto-extractions and analysis were carried out at regular time intervals during the course of the size-reduction process. Chromatograms from the MALS and RI detectors are shown in FIGS. 6A and 6B respectively. FIG. 7 follows the size-reduction process of the polysaccharide during the course of the size-reduction process and shows a continuous decrease in average polysaccharide molecular weight over time.

### Example 5

### At-Line LC-MALS-RI Procedure

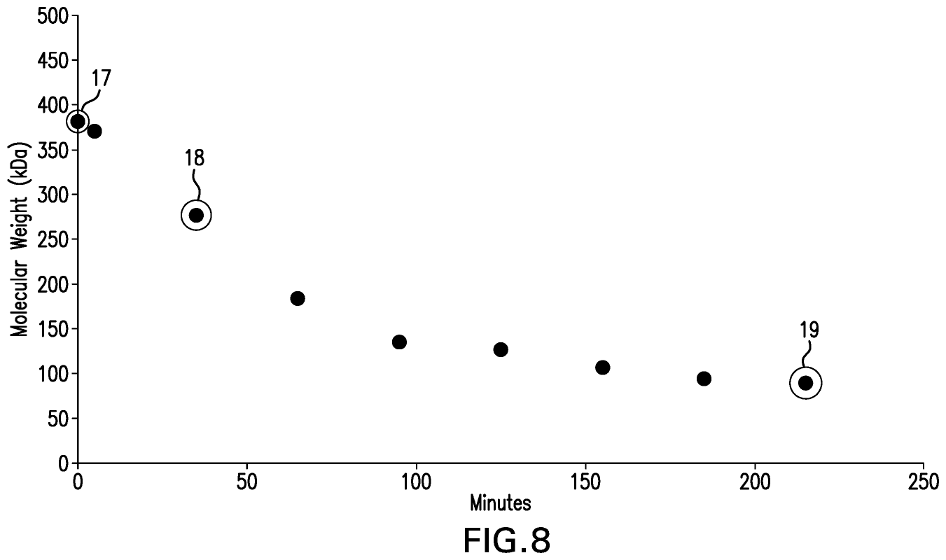

A solution of S. pneumoniae Serotype 6A in acetic acid (200 mM, solution concentration about 3.96 mg/mL) solution was introduced into a jacketed vessel having a jacket temperature of 80° C. and stirring was commenced to initiate an acid-hydrolysis based size reduction process. At initiation, and throughout the size-reduction process, 1.5 mL aliquots were taken manually from the vessel, quenched with phosphate buffer, and injected into an At-line LC-MALS-RI system for analysis. The At-line LC-MALS-RI system encompassed a Waters Patrol UPLC system (the “LC system”), a Wyatt uDawn MALS detector (the “MALS detector”), and a Wyatt Optilab UT-rEX RI detector(“RI detector”), which were connected in sequence. Each aliquot was manually injected directly into the LC system, and chromatographically separated using size exclusion chromatography (SEC) (flowrate for the SEC method was 0.5 mL/min, the temperature of the column was designated at 30° C., and the mobile phase was 10 mM BisTris and 150 mM NaCl buffer, at pH 6.8). Upon exiting the SEC column, the chromatography eluate was subsequently introduced into the MALS detector, where the multi-angle light scattering of the polysaccharides was measured. Upon exiting the MALS detector, the sample was introduced into the RI detector, where the refractive index of the polysaccharides was measured. Using Wyatt Astra software, the light scattering and refractive index signals were utilized to calculate the average molecular weight of the polysaccharides in the sample. Manual sample extractions from the size-reduction process and were carried out at regular time intervals during the course of the size-reduction process and injected into the LC system. FIG. 8 shows the progression of follows the size-reduction process of the polysaccharide during the course of the size-reduction process and shows a continuous decrease in average polysaccharide molecular weight over time.

1. A method for monitoring the size-reduction of polysaccharides, wherein said method comprises the steps:
i) subjecting the polysaccharides to a size-reduction process, wherein the polysaccharides are in an aqueous solution during the size-reduction process;
ii) transferring a sample of the aqueous solution from step i to an instrument that can analyze the molecular weight of the polysaccharides in the aqueous solution;
iii)measuring the average molecular weight of the polysaccharides in the sample using the instrument; and
iv) optionally subjecting a subsequent sample of the aqueous solution from step i, to steps ii, iii, and iv.

2. A method for monitoring the size-reduction of polysaccharides, wherein said method comprises the steps:
i) subjecting the polysaccharides to a size-reduction process, wherein the polysaccharides are in an aqueous solution during the size-reduction process;
ii) transferring a sample of the aqueous solution of step i to a liquid chromatography system that performs a chromatographic separation of the polysaccharides in the sample; then
iii) transferring the chromatography eluent of step ii to a first instrument that measures the light scattering of each of the polysaccharide components in the sample;
iv) transferring the sample solution exiting the first instrument to a second instrument, wherein the second instrument measures the concentration of each of the polysaccharide components using the refractive index of the polysaccharides;
v) calculating the average molecular weight of the polysaccharides in the aqueous solution, wherein the method of calculation utilizes: (a) the light scattering information obtained in step iii, and (b) the refractive index information obtained in step iv; and
vi) optionally subjecting a subsequent sample of the aqueous solution from step i to steps ii, iii, iv, v and vi.

3. The method of claim 1, wherein the aqueous solution of step i further comprises a salt, or wherein a salt is added to the sample after step i, prior to commencing step ii.

4. (canceled)

5. The method of claim 1, wherein the polysaccharides are bacterial surface polysaccharides.

6. The method of claim 5, wherein the bacterial surface polysaccharides are derived from pneumococcal bacteria.

7. The method of claim 1, wherein the size-reduction process comprises a physical method.

8. (canceled)

9. The method of claim 1 wherein the size-reduction process comprises a chemical method.

10. (canceled)

11. The method of claim 1, wherein the measuring in step iii is conducted using high-performance size exclusion chromatography, multi-angle light scattering, or refractive index.

12. (canceled)

13. The method of claim 1, wherein the method is repeated in its entirety from 1 to 40 times.

14. The method of claim 13, wherein the method is repeated in its entirety from 3 to 10 times.

15. (canceled)

16. The method of claim 1, wherein size reduction process is carried out for a total time of from about 1 hour to about 6 hours.

17. The method of claim 1, wherein the transferring of step ii is repeated at 2 second intervals.

18. The method of claim 1, wherein the measuring of step iii is done continuously in real time.

19. The method of claim 1, wherein the sample of the aqueous solution from step i is filtered prior to commencing step ii.

20. (canceled)

21. A conjugate immunogenic composition comprising: i) the size-reduced bacterial surface polysaccharides obtained according to the method of claim 1, and ii) a protein carrier, wherein the final average bacterial surface polysaccharide distribution is from 70-1000 kDa.

22. (canceled)

23. The conjugate immunogenic composition of claim 21, wherein the immunogenic composition comprises the purified capsular polysaccharide of 21 serotypes of Streptococcus pneumoniae, wherein the serotypes are 3, 6A, 7F, 19A, 22F, 33F, 8, 10A, 11A, 12F, 15B, 15A, 23B, 24F, 35B, 16F, 17F, 20, 23A, 31, and 9N, wherein the 15B is de-O-acetylated.

24. The conjugate immunogenic composition of claim 21, wherein the immunogenic composition comprises the purified capsular polysaccharide of 15 serotypes of Streptococcus pneumoniae, wherein the serotypes are 4, 6B, 9V, 14, 18C, 19F, 23F, 1, 3, 5, 6A, 7F, 19A, 22F and 33F.

25. The conjugate immunogenic composition of claim 21, wherein the immunogenic composition comprises the purified capsular polysaccharide of 24 serotypes of Streptococcus pneumoniae, wherein the serotypes are 4, 6B, 9V, 14, 18C, 19F, 23F, 1, 3, 5, 6A, 7F, 19A, 22F, 33F, 8, 10A, 11A, 12F, 15B, 15A, 23B, 24F, and 35B wherein the 15B is de-O-acetylated.

26. The conjugate immunogenic composition of claim 21, wherein the immunogenic composition comprises the purified capsular polysaccharide of 20 serotypes of Streptococcus pneumoniae, wherein the serotypes are 4, 6B, 9V, 14, 18C, 19F, 23F, 1, 3, 5, 6A, 7F, 19A, 22F, 33F, 8, 10A, 11A, 12F, and 15B.

27. A method for treating or preventing a disease in a patient, wherein the disease is caused by Streptococcus pneumoniae, wherein the method comprises administering an effective amount of the conjugate immunogenic composition of claim 21 to the patient.
